(19) United States
(12) Patent Application Publication
(10) Pub. No.: US 2010/0120127 A1
(43) Pub. Date: May 13, 2010

(54) METHOD FOR REMOVING BACTERIA FROM A FERMENTATION BROTH

(75) Inventors: Kevin O'Mahony, Lausanne (CH); Frank Hilbrig, Bayreuth (DE); Ruth Freitag, Bayreuth (DE); Ivo Schumacher, Jona (CH)

Correspondence Address: MICHAEL J. STRIKER, 103 EAST NECK ROAD, HUNTINGTON, NY, 11743, US

(73) Assignees: DRM, DR. MÜLLER AG, Männedorf (CH); POLYTAG AG, Männedorf (CH)

(21) Appl. No.: 11/720,663

(22) PCT Filed: Nov. 7, 2005
(86) PCT No.: PCT/CH2005/000649

§ 371 Date: Jun. 1, 2007

(30) Foreign Application Priority Data
Nov. 15, 2004 (CH) 1870/04

(51) Int. Cl. C12N 1/02

(52) U.S. Cl. 435/261

(57) ABSTRACT

The invention relates to method for the stepwise removal of bacteria from a fermentation broth. According to said method, the bacteria in the fermentation broth are mixed with a retention promoter mixture in a first process step and the pretreated bacteria are removed by precoat filtration in a second process step. The inventive method uses a retention promoter mixture from bentonite and polycations such as PEI or polyDADMAC and is suitable for treating large quantities of bacteria. The filter cake obtained with bentonite/PEI has a substantially improved quality.

The present invention relates to a process for stepwise removal of bacteria from a fermentation broth.

Precoat filtration is a known process for removing solids from liquids and gases. It is particularly important in biotechnical processes where cell material from a culture broth of a fermenter must be removed.

A process for removing yeast cells by precoat filtration is known from GB 1,082,862. In this patent are mentioned filter aids such as starch and silicate-containing fuller's earths as well as cellite. The behavior of the relatively large yeast cells is different from that of bacteria.

A candle precoat filter of this kind is known from EP 0,064,795. The known filter is used for continuous removal of nonspecific waste-water sludges. Filter aids are not mentioned.

WO 01/81249 A1 discloses a system for purifying water or gases of chemical or microbiological impurities. The microorganisms mentioned are bacteria or viruses. The known system consists of a container which can hold a bentonite retention agent, a polymer and some other material. Bentonite is known to be able to fix bacteria, viruses etc. For this reason, it is proposed that it can also remove bacteria from a ventilation system. The known method makes use of a polymer. Bentonite is fixed on a material in the container. Filtration with filter aids, namely precoat filtration, is not feasible.

According to U.S. Pat. No. 5,453,200, in the culturing of microorganisms these are isolated by filtration or centrifugation. After the removal of the microorganisms, it is proposed to treat the supernatant filtrate with bentonite to absorb the protein contaminants. In this case, therefore, bentonite is not used to improve the retention of the microorganisms.

EP 0391 253 describes a process for purifying and deodorizing enzyme solutions, particularly for removing troublesome colored and odoriferous contaminants, with the aid of absorption agents, among others also bentonite. Bentonite is not used for the removal of production organisms.

The object of the invention is to provide a process making possible an efficient and possibly complete removal of bacterial biomass from fermentations at biomass concentrations that are as high as possible. It is to be assumed that the isolated bacterial cells will serve as base material for further processing.

According to the invention, this objective is reached in that in the first processing step the bacteria-containing fermentation broth is mixed with a retention promoter and in a second processing step the pretreated bacteria are removed by precoat filtration. Suitable retention promoters are mixtures of a polycation [poly(ethyleneimine) (PEI), poly(diallyldimethylammonium chloride) (polyDADMAC)] and a multilayer silicate (bentonite) as well as modified (positively charged) celluloses.

At the time of the precoat filtration, the biomass concentration should correspond to an optical density OD₆₀₀ of 15. When necessary, this can be achieved by dilution. If cultures with higher optical densities are filtered without a previous dilution step, the efficiency of cell retention and of the pressure drop above the filter cake increase markedly.

The precoat filtration is carried out at a flow rate of 500 to 1500 L/(m².h).

A particularly advantageous retention promoter mixture is one containing from 5 to 500 mg/L of polycation (PEI, polyDADMAC) and 2 to 10 g/L of bentonite. These conditions ensure a nearly complete bacterial retention (at least >98%). If lower amounts of polycation and/or bentonite are added, the efficiency of bacterial retention drops (<50%); higher amounts of polycations do not improve the filtration efficiency any further and lead to higher filtration pressures, whereas with higher amounts of bentonite added (more than 10 g/L) the filter cakes are unnecessarily voluminous leading to uneconomical filtration conditions. At the same time the filtration pressures also rise.

Bentonite is a multilayer silicate also known under the synonyms smectite and montmorillonite.

It has been found advantageous to use bentonite activated with sodium or sodium bentonite. Bentonite activated with sodium has given particularly good results. Calcium bentonite was also found to be useful.

Other outstandingly well suited polycations for the retention promoter mixture are poly(ethyleneimine) (PEI) made by different manufacturers. PEI is a polycation derived from ethyleneimine units. Among these, high-molecular-weight PEI (1000-50,000 g/mol) are better suited than low-molecular-weight ones (<1000 g/mol), and for comparable molecular weights branched molecules are better suited than unbranched ones. A second polycation that has been proven to be well suited as retention promoter is poly(diallyldimethylammonium chloride) (polyDADMAC).

Whereas the first processing step (treatment with the retention promoter mixture) is carried out under processing conditions, it is advantageous just before the filtration and before adding the filter aid to increase the pH of the suspension to a value of 8, preferentially by addition of an appropriate amount of tris-buffer.

It is also advantageous to stir in 100-1000 mM of NaCl just before the filtration. The addition of tris and NaCl has the advantage that the filtration pressure and, hence, the permeability of the filter cake are slightly increased.

Bacteria (compared to eukaryotic cells such as yeasts, molds or animal/plant cells) are very small organisms used for production in biotechnology in many different ways. Their complete removal from the culture supernatant by simple filtration without filter aids is impossible, and the removal by precoat filtration involves extremely voluminous, mechanically stable filter cakes (inclusion of the bacteria into the cake) which in the event of cell densities commonly prevailing in bioproduction would require filter aid quantities orders of magnitude higher than commonly viewed as industrially compatible. It is not possible to reduce substantially the required amounts of filter aids by pretreatment of the bacteria [for example, with polycations (for example PEI, polyDADMAC)], multiply charged metal ions (for example Fe³⁺, Al³⁺, Ca²⁺), charged adsorbents such as positively charged cellulose or by crosslinking with negatively charged silicate particles). At the same time, in all cases the fixation of the cells by the filter cake is not sufficient for obtaining a robust process. Typically, the bacteria are again washed out of the filter cake even by low fluctuations in pressure or volume flow (leaching of bacteria). This makes it impossible to process bacteria in the filter cake (washing, buffer exchange, cell lysis) and thus to develop any arrangement for process intensification by process integration.

Surprisingly, the use of bentonite and polycations such as PEI or polyDADMAC as retention promoters for precoat filtration leads to a complete and entirely stable removal of bacteria in filter cakes of a manageable size. The observed high efficiency of bacterial retention has been proven to occur by way of a pure fixation effect of the retention promoter mixture and probably is attributable to the formation of a hydrogel by the bentonite during the incubation phase. At any rate, a bacterial filtration following the treatment of the culture with PEI (polyDADMAC)/bentonite is basically of a different quality (pressure drop, filtrability, bacterial retention, fixation of bacteria in the filter cake) than is the otherwise analogous filtration (filter aid, flow rate) without pretreatment or after pretreatment with a polycation.

### EXAMPLE FOR BACTERIA

Bacteria (strain: E. coli DH5 from Clontech-BD Biosciences, Palo Alto, Calif., USA) were cultivated in the known manner in a bioreactor (Chemap FZ). The culture medium (pH 6.8) consisted of at least 10 g/L of glucose, 2 g/L of NaCl, 1.2 g/L of MgSO₄, 6 g/L of Na₂HPO₄, 3 g/L of KH₂PO₄, 3 g/L of (NH₄)₂SO₄, 1.7 g/L of citric acid, 0.01 g/L of CaCl₂ and trace elements (FeCl₃: 60 mg/L, Na₂EDTA: 8.4 mg/L, Zn(CH₃COO)₂: 8.0 mg/L, MnCl₂: 15.0 mg/L, CoCl₂: 2.5 mg/L, Na₂MoO₄: 2.5 mg/L, CuCl₂: 1.5 mg/L, H₃BO₃: 3.0 mg/L). O₂ was not measured, but the cultivation was carried out at maximum air flow, namely about 30 L/min. The temperature was set at 37° C. After 16 hours, the bioreactor was ready for harvesting (OD₆₀₀>15).

The filtration was carried out with the aid of a laboratory candle filter (type TSD-0.012 m², supplied by the DrM company, Dr. Müller AG, Männedorf, Switzerland). The laboratory candle filter had a surface area of 75 cm² or 40 cm² and depending on the biomass concentration allowed a filtration of a few liters of cell suspension per batch. A membrane (B31M30, G11M200 or G11M080 all supplied by DrM) supported by a metallic plate was used as filter medium. Different filter aids (diatomaceous earths) were used, among others the “Celpure” materials supplied by World Minerals Inc. (Advanced Minerals and Cellite), St. Barbara, Calif., USA, and the “Celatom” materials supplied by Eagle Pisher Filtration and Minerals Europe.

The experiments were carried out under typical industrial conditions. The flow rate was adjusted to between 500 and 1500 L/m².h). The amount of filter aid was between 10 and 20 g/L. Unless otherwise indicated, all experiments were carried out with bacterial cultures of E. coli of the nominal OD₆₀₀ of 15.

OD=optical density. The measurement is carried out in the known manner with light of a wavelength of 600 nm and indicates the turbidity of the solution. It gives the experimental value of the concentration of the bacteria in the suspension.

Experiments with different kinds, particle sizes and amounts of filter aids such as kieselguhr (diatomaceous earth) were unsuccessful. Only uneconomically large amounts of kieselguhr produced a usable clarification. An effective and economical removal of the bacteria of a culture with filter aids alone was not possible. Moreover, the bacteria were not retained in the filter cake with sufficient stability. This makes the filtration unreliable and affects negatively or prevents altogether any further treatment (washing, cell lysis) of the bacteria-containing filter cake.

The experimental results are shown in Tables 1 and 2.

TABLE 1
Results of the Body Feed Filtration Experiments
with Various Diatomaceous Earths as Filter Aids

| Amounts and Type of | | |
|---|---|---|
| Filter Aid | Final Pressure | Bacteria Retention*⁾ |
| 5 g/L of Celpure P100 | 3 bar** | 72% |
| 10 g/L of Celpure P100 | 3 bar** | 65% |
| 5 g/L of Celpure P300 | 2.85 bar** | 36% |
| 10 g/L of Celpure P300 | 0.8 bar | 38.5% |
| 15 g/L of Celpure P300 | 0.6 bar | 50% |
| 20 g/L of Celpure P300 | 0.8 bar | 74% |
| 30 g/L of Celpure P300 | 0.4 bar | 75% |
| 0.25 g/L of Celpure P1000 | 1.8 bar | 13% |
| 1 g/L of Celpure P1000 | 0.6 bar | 16% |
| 5 g/L of Celpure P1000 | 0 bar | 19% |
| 10 g/L of Celatom FW 12 | 0.9 bar | 69% |
| 30 g/L of Celatom FP 4 | 1.35 bar | 66% |

*⁾Always refers to the amount retained in the cake in regard to the volume;
**The filtration had to be terminated prematurely due to clogging of the filter cake.

TABLE 2
Results of the Body Feed Filtration Experiments with Mixtures
of Fine and Coarse Diatomaceous Earths as Filter Aids

| Ratio P1000:P100, Total Amount | Final Pressure | Bacteria Retention |
|---|---|---|
| P1000 alone (5 g/L) | 0.05 bar | 23% |
| 9:1.2 g/L | 2.6 bar* | 9% |
| 9:1.5 g/L | 1.1 bar | 23% |
| 9:1.10 g/L | 0.3 bar | 23% |
| 9:1.15 g/L | 0.2 bar | 39% |
| 3:1.2 g/L | 2.5 bar* | 19% |
| 3:1.5 g/L | 1.2 bar | 27% |
| 3:1.10 g/L | 0.4 bar | 33% |
| 3:1.15 g/L | 0.2 bar | 43% |
| 1:1.5 g/L | 1.5 bar | 53% |
| 1:1.10 g/L | 0.6 bar | 37% |
| 1:4.5 g/L | 3.0 bar* | 55% |
| 1:4.10 g/L | 1.5 bar | 62% |
| Celatom FE6:FW12, 1:1.10 g/L | 2.4 bar* | 82% |

*Filtration had to be terminated prematurely due to clogging of the filter cake.

Filtration experiments with other filter aids (cellulose, J. Rettenmaier & Sons, Rosenberg, Germany, no retention observed) or after flocculation of the bacteria with polycations (PEI, polyDADMAC) or with multiply positively charged metal ions (Fe³⁺, Al³⁺, Ca²⁺), Table 3, or by adsorption on positively charged cellulose, Table 4, and crosslinking by positively charged ions (polycations, metal ions) with negatively charged filter aid particles, Table 5, were unsuccessful in terms of as complete as possible but above all stable retention of the bacteria in a filter cake of industry-compatible size. Note at any rate that after the filtration the bacteria adsorbed on positively charged cellulose (also provided by J. Rettenmaier & Sons) (retention as a rule <50% of the “industry-compatible” maximum filter aid amounts) were stably established in the filter cake and that such filter cakes could be washed, for example without appreciable leaching of the bacteria, with different washing buffers. This has not been observed in any of the previously reported experiments.

TABLE 3
Results of the Filtration Experiments After Flocculation with PEI

| | | Bacteria |
|---|---|---|
| Conditions | Final Pressure | Retention |
| FW 6 10 g/L, PEI 3, 50 mg/L | 1.6 bar | 81% |
| FW 12 10 g/L, PEI 3, 50 mg/L | 1.1 bar | 77% |
| FW 12 10 g/L, PEI 3, 25 mg/L, | 3.0 bar | 86% |
| pH 8, 50 mM NaCl | | |
| FW 12 20 g/L, PEI 3 25 mg/L, pH 8, | 1.7 bar | 87% |
| 50 mM NaCl | | |
| FW 12 20 g/L, PEI 3 37.5 mg/L, pH 8, | 1.5 bar | 87.5% |
| 50 mM NaCl | | |
| FW 12 10 g/L, PEI 4 5 mg/L | 2.0 bar | 87% |
| FW 12 10 g/L, PEI 4 25 mg/L | 1.4 bar | 94% |
| FW 12 10 g/L, PEI 4 150 mg/L | 2.0 bar | 100% |
| FW 14 10 g/L, PEI 3 25 mg/L, pH 8, | 2.2 bar | 83% |
| NaCl 50 mM | | |
| FW 14 20 g/L, PEI 3 37.5 mg/L, pH 8 | 1.5 bar | 87% |
| FW 14 20 g/L, PEI 3 37.5 mg/L, | 1.2 bar | 86% |
| pH 8, NaCl 50 mM | | |
| FW 20 10 g/L, PEI 3 25 mg/L, pH 8, | 1.5 bar | 74% |
| NaCl 50 mM | | |
| FW 20 10 g/L, PEI 4 50 mg/L | 3.0 bar | 70% |
| FW 20 20 g/L, PEI 4 50 mg/L | 2.6 bar | 92% |

*) Filtration had to be terminated prematurely due to clogging of the filter cake.

TABLE 4
Capture/Sedimentation of Bacteria with Charged Cellulose

| | | | | FDY | FIC | EF | |
|---|---|---|---|---|---|---|---|
| | BE 600/20 | BE 600/20 | BE 600/20 | 600 | 200 | 900 CT | EFC |
| Conditions | 10 g/L | 5 g/L | 1 g/L | 10 g/L | 10 g/L | 10 g/L | 950 C 10 g/L |
| pH 5.5 | | | | | | | |
| 0.0 M NaCl | 31% | | | 27% | 28% | 23% | 24% |
| 0.1 M NaCl | 23% | 17% | 12% | | | | |
| 0.25 M NaCl | 23.5% | 18% | 11% | | | | |
| 0.5 M NaCl | 28% | 22.5% | 13% | | | | |
| pH 7.0 | | | | | | | |
| 0.0 M NaCl | 29% | | | 20% | 27% | 21% | 18% |
| 0.1 M NaCl | 25% | 22% | 18% | | | | |
| 0.25 M NaCl | 30% | 23% | 16% | | | | |
| 0.5 M NaCl | 28% | 17% | 18% | | | | |
| pH 8.0 | | | | | | | |
| 0.0 M NaCl | 29% | | | 17% | 24.5% | 20% | 17% |
| 0.1 M NaCl | 36% | 34% | 28% | | | | |
| 0.25 M NaCl | 38.5% | 29% | 27% | | | | |
| 0.5 M NaCl | 37% | 29% | 28% | | | | |

TABLE 5
Filtration of Bacteria Crosslinked to Diatomaceous Earth

| | Final | Bacteria |
|---|---|---|
| Conditions | Pressure | Retention |
| 10 g/L FW 12, 10 mg/L AICl₃ | 3.0 bar* | 75.5% |
| 10 g/L FW 12, 1 mg/L AICl₃ | 3.5 bar* | 74% |
| 10 g/L FW 12, 0.1 mg/L AICl₃ | 2.0 bar | 69% |
| 10 g/L FW 12, 5 mg/L PEI 4 | 2.0 bar* | 87% |
| 10 g/L FW 12, 25 mg/L PEI 4 | 1.4 bar* | 94% |
| 10 g/L FW 12, 150 mg/L PEI 4 | 2.0 bar* | 100% |
| 10 g/L P1000, 75 mg/L PEI 4 | 1.1 bar | 88% |
| 10 g/L P1000, 100 mg/L PEI 4 | 1.3 bar | 97% |
| 10 g/L P1000, 125 mg/L PEI 4 | 1.2 bar | 95% |
| 10 g/L FW 20, 50 mg/L PEI 4 | 3.0 bar | 70% |
| 20 g/L FW 20, 50 mg/L PEI 4 | 2.6 bar | 92% |
| 10 g/L FW 12, 10 g/L P1000, 5 mg/L PEI 4 | 0.6 bar | 75% |
| 10 g/L FW 12, 10 g/L P1000, 25 mg/L PEI 4 | 0.4 bar | 83% |
| 10 g/L FW 12, 10 g/L P1000, 150 mg/L PEI 4 | 1.5 bar | 90.5% |

*Filtration had to be terminated prematurely due to clogging of the filter cake.

### EXPERIMENTS WITH BENTONITE

Bentonite is a multilayer silicate which in contact with aqueous solutions swells more or less strongly. This swelling leads to an absorption of different substances including bacteria from the surroundings. After numerous preliminary experiments, bentonite activated with sodium was selected for carrying out the practical example. The mineral swells to an extent that lies between that of sodium bentonite, which swells strongly, and calcium bentonite which swells only very slightly. The mineral is positively charged in the spaces between the intermediate layers. This allows an electrostatic interaction with the negatively charged bacteria. Hence, theoretically the mineral has the ability to bind bacteria both by adsorption and by absorption.

The results of numerous experiments with precoat filtration of bacteria treated with bentonite are summarized in Table 6. Included here are experiments carried out with pretreatment of the bacterial suspension with bentonite alone as well as those in which besides bentonite multiply charged metal ions or polycations were used for the pretreatment. Whereas the treatment with bentonite alone, particularly in the case of cultures with a low biomass (OD₆₀₀<15) and minimal media (LB-Medium), occasionally led to success, a robust and reliable process could be developed only by treatment (incubation) with the bentonite/polycation mixture.

TABLE 6
Filtration of Bacterial Suspension in the Presence of Bentonite

| | Final | Bacteria |
|---|---|---|
| Conditions | Pressure | Retention |
| 20 g/L FW 12, 5 g/L WGA, LB-Medium* | 1.4 bar | 93% |
| 20 g/L FW 14, 5 g/L WGA, LB-Medium* | 3 bar | — |
| 20 g/L FW 14, 5 g/L WGA, LB-Medium* | 1.3 bar | — |
| 20 g/L FW 14, 5 g/L WGA, LB-Medium*, | 0.2 bar | 52% |
| 250 mM NaCl | | |
| 20 g/L FW 14, 5 g/L WGA, LB-Medium*, | 0.3 bar | 53% |
| 500 mM NaCl | | |
| 20 g/L FW 14, 5 g/L WGA, LB-Medium, 50 mM | 0.5 bar | 55% |
| tris pH 8, 250 mM NaCl | | |
| 20 g/L FW 50, 5 g/L CV, LB-Medium* | 1.3 bar | 94% |
| 20 g/L FW 14, 5 g/L WGA, 50 mM tris pH 8, | 0.4 bar | 69% |
| 250 mM NaCl, 0.1 g/L FeCl₃ | | |
| 20 g/L FW 14, 5 g/L WGA, 50 mM tris pH 8, | 1.4 bar | 89% |
| 250 mM NaCl, 0.2 g/L FeCl₃ | | |
| 20 g/L FW 14, 5 g/L WGA, 50 mM tris pH 8, | 1.8 bar | 93% |
| 250 mM NaCl, 0.3 g/L FeCl₃ | | |
| 20 g/L FW 14, 5 g/L WGA, 50 mM tris pH 8, | 2.2 bar | 92% |
| 250 mM NaCl, 0.4 g/L FeCl₃ | | |
| 20 g/L FW 14, 5 g/L WGA, 50 mM NaCl, | 0.4 bar | 65% |
| 50 mg/L PEI 4 | | |
| 20 g/L FW 14, 5 g/L WGA, 50 mM tris pH 8, | 2.1 bar | 100% |
| 50 mg/L PEI 4 | | |
| 20 g/L FW 14, 5 g/L WGA, 50 mM tris pH 8, | 1.4 bar | 100% |
| 250 mM NaCl, 50 mg/L PEI 4 | | |
| 20 g/L FW 14, 5 g/L WGA, 50 mM tris pH 8, | 0.6 bar | 98% |
| 1000 mM NaCl, 50 mg/L PEI 4 | | |
| 20 g/L FW 14, 5 g/L WGA, 50 mM tris pH 8, | 0.6 bar | 100% |
| 250 mM NaCl, 100 mg/L PEI 4 | | |
| 20 g/L FW 14, 5 g/L WGA, 50 mM tris pH 8, | 0.2 bar | 58% |
| 250 mM NaCl, 50 mg/L polyDADMAC | | |
| 20 g/L FW 14, 5 g/L WGA, 50 mM tris pH 8, | 0.9 bar | 100% |
| 250 mM NaCl, 150 mg/L polyDADMAC | | |
| 20 g/L FW 14, 5 g/L WGA, 50 mM tris pH 8, | 0.9 bar | 100% |
| 250 mM NaCl, 50 mg/L PEI 4 | | |
| 0 g/L FW 14, 5 g/L WGA, 50 mM tris pH 8, | 3 bar | 100% |
| 250 mM NaCl, 50 mg/L PEI 4** | | |

*Filtration terminated prematurely due to clogging of the filter cake;
**Repetition of the experiment above with a culture containing much debris.

Surprisingly, the filtration of the bacteria fixed in this manner led to nearly 100% retention. Leaching of the bacteria (recognizable by a clearly visible increase in turbidity of the filtrate) was not observed even after applying a pressure pulse during such filtrations.

The efficacy of bentonite cannot be explained on the basis of adsorption/absorption alone. A synergistic effect appears to be involved which obviously improves the retention of the bacteria. This effect is probably caused by the formation of hydrogels induced by the bentonite. Best results are observed at pH 8, probably because here the positive charge of PEI is sufficiently pronounced for a stable interaction with the negatively charged bacteria. In experiments without polycation addition, a decrease in pH to values between 5.0 and 5.5, improved the process in that, under otherwise comparable conditions, the filtration pressure required was reduced. This is probably attributable to a degradation of the pH-sensitive hydrogels. Consistent with this is also the observation that after a decrease in pH such suspensions must be further processed as soon as possible, because otherwise the retention of the bacteria and their stability in the filter cake decrease. The addition of up to 1 M NaCl also reduces the pressure needed for the filtration without causing an appreciable deterioration in bacterial retention/stability.

Overall, however, these effects are observed primarily with filtrations without polycation addition. If the bacteria are filtered after pretreatment with bentonite/polycation at pH 8, the process is very robust, and other parameters such as temperature, salt content or media composition play hardly any role in achieving success.

### PRACTICAL EXAMPLE

At the end of the bacterial culture, the contents of the bioreactor (fermenter) were treated with 5/g/L of bentonite and 50 mg/L of PEI and the mixture was stirred at room temperature for 1 hour. By addition of a suitable tris-buffered solution, the bacterial suspension was then brought to a nominal biomass concentration of not more than OD₆₀₀=15, a pH of 8, a NaCl concentration of 250 mM and a tris concentration of 50 mM. The filter aid was then added (for example 20 g/L of Celatom FW 14), and the mixture was quickly filtered at a flow rate of 600 L/(m².h). One filtration was carried out with added bentonite and one without.

The average values of the results are presented graphically. We can see

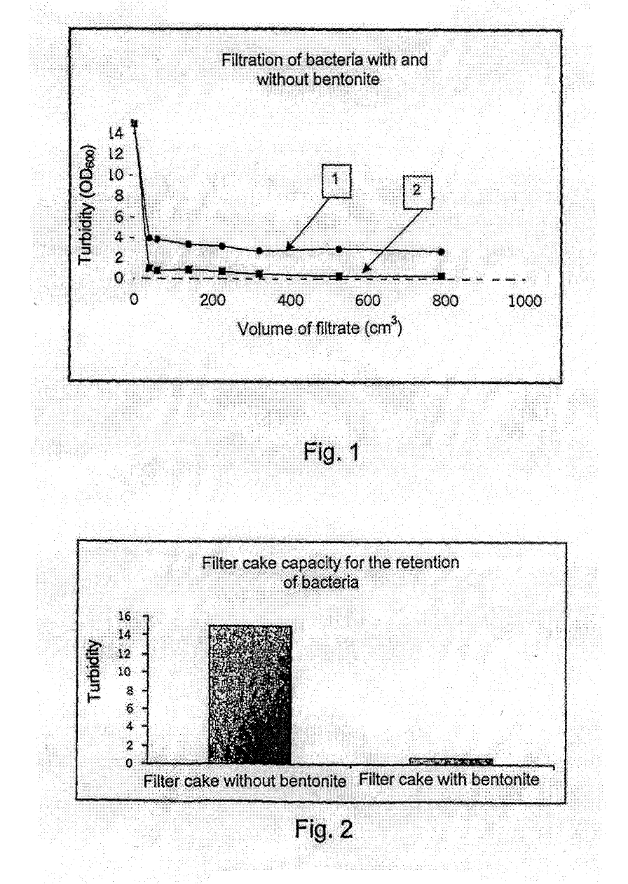

in FIG. 1 a comparison of filtration with and without bentonite,

in FIG. 2 the retention capacity of the bacteria in the filter cake with and without bentonite.

In FIG. 1, the curve shows the filtration without bentonite; curve 2 shows the filtration with addition of bentonite. In the latter case, surprisingly, the bacterial retention amounted to 95-100% as can be seen from the fact that the turbidity approaches zero.

The filtration by use of bentonite gives a clear filtrate depending on the type of bentonite used. To achieve the same effect with known filter aids alone, one order of magnitude higher amounts of filter aid would be needed.

FIG. 2 shows the results after washing the filter cake. A washing buffer placed on the filter cake at high pressure and without bentonite would cause detachment of the bacteria from the cake (left bar). The bacteria are not firmly linked with the cake. A filter cake with bentonite, on the other hand, can be washed without loss of bacteria (right bar). This indicates that the filter cake obtained with bentonite is of substantially better quality.

A great advantage of the process of the invention is that it is eminently suited for treating large amounts of bacteria, because a permeable, stable filter cake is attained by use of bentonite. The filter cake can be readily washed with other liquids or subjected to additional processing steps such as in-situ cell lysis. Moreover, at the end of the filtration such a filter cake can easily be air-dried. Furthermore, at the end of the filtration the filter cake can readily be removed from the filter elements, for example a candle filter. Sticking to the filter media does not take place.

1. Process for stepwise removal of bacteria from a fermentation broth, characterized in that in a first processing step the bacteria of the fermentation broth are incubated with a retention-promoting mixture and in the second processing step the pretreated bacteria are removed by precoat filtration.

2. Process as defined in claim 1, characterized in that the precoat filtration is carried out at a flow rate of 500 to 1500 l/(m².h).

3. Process as defined in claim 1, characterized in that between 2 and 20 g/L of filter aid is used for the precoat filtration.

4. Process as defined in claim 1, characterized in that the precoat filtration is carried out with a kieselguhr (diatomaceous earth) as filter aid.

5. Process as defined in claim 1, characterized in that the fermentation broth is incubated with the retention-promoting mixture for one hour.

6. Process as defined in claim 1, characterized in that the retention-promoting mixture contains from 2 to 20 g/L of bentonite.

7. Process as defined in claim 1, characterized in that the bentonite is sodium bentonite.

8. Process as defined in claim 1, characterized in that the bentonite is calcium bentonite.

9. Process as defined in claim 1, characterized in that the retention-promoting mixture contains from 0.5 to 500 mg/L of PEI.

10. Process as defined in claim 1, characterized in that the PEI contained in the retention-promoting mixture has a molecular weight between 1000 and 50,000 g/mol.

11. Process as defined in claim 1, characterized in that the retention-promoting mixture contains a branched PEI.

12. Process as defined in claim 1, characterized in that the retention-promoting mixture contains from 0.5 to 500 mg/L of polyDADMAC.

13. Process as defined in claim 1, characterized in that just before the filtration the pH of the suspension is adjusted to a value of 8.

14. Process as defined in claim 1, characterized in that just before the filtration 50-1000 mM of NaCl is mixed in.

15. Process as defined in claim 1, characterized in that just before the filtration the buffer concentration is adjusted to 50 mM.

16. Process as defined in claim 1, characterized in that just before the filtration the buffer concentration is adjusted to 50 mM tris.

17. Process as defined in claim 1, characterized in that the concentration of the bacteria in the fermentation broth has an optical density OD₆₀₀ of at the most 15.

18. Process as defined in claim 1, characterized in that the nominal concentration of the bacteria in the fermentation broth after the incubation with the retention promoter and just before the filtration is adjusted (diluted) to a value of OD₆₀₀=15.

19. Process as defined in claim 1, characterized in that the nominal OD₆₀₀, the pH, the NaCl concentration and the buffer concentration together are adjusted with a suitable tris-buffered solution.

20. Process as defined in claim 1, characterized in that the retention promoter used is a modified (positively charged) cellulose.
